(19) United States
(12) Patent Application Publication
(10) Pub. No.: US 2004/0219106 A1
(43) Pub. Date: Nov. 4, 2004

(54) Compounds for use as surfactants

(76) Inventors: Brian Edgar Looker, Stevenage (GB); Christopher Lunniss, Stevenage (GB); Alison Redgrave, Stevenage (GB)

Correspondence Address: DAVID J LEVY, CORPORATE INTELLECTUAL PROPERTY, GLAXOSMITHKLINE, FIVE MOORE DR., PO BOX 13398, RESEARCH TRIANGLE PARK, NC, 27709-3398, US

(21) Appl. No.: 10/484,958

(22) PCT Filed: Jul. 24, 2002
(86) PCT No.: PCT/GB02/03375

(30) Foreign Application Priority Data
Jul. 27, 2001 (GB) 0118364.9

(51) Int. Cl. A61L 9/04; C07F 9/02

(52) U.S. Cl. 424/45; 554/80

(57) ABSTRACT

A compound of formula (I) or a salt thereof, wherein: R^{1a} represents C₁₋₃ alkyl; R^{1b} represents C₁₋₃ alkyl; R^{1c} represents C₁₋₃ alkyl; R²represents C₁₋₅ fluoroalkyl; R³represents C₁₋₅ fluoroalkyl; X represents —C₁₋₆ alkylene-; Y represents —C₁₋₆ alkylene-; with the proviso the each C₁₋₅ fluoroalkyl contains 3 or fewer consecutive perfluorocarbon atoms, methods of preparing said compounds, pharmaceutical aerosol formulation comprising said compounds and uses thereof.

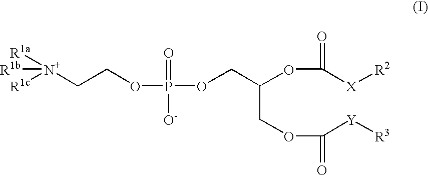

This invention relates to aerosol formulations of use for the administration of medicaments by inhalation and to compounds having surfactant properties for use therein.

The use of aerosols to administer medicaments has been known for several decades. Such aerosols generally comprise the medicament, one or more chlorofluorocarbon propellants and either a surfactant or a solvent, such as ethanol. The most commonly used aerosol propellants for medicaments have been propellant 11 (CCl₃F) and/or propellant 114 (CF₂ClCF₂Cl) with propellant 12 (CCl₂F₂). However, these propellants are now believed to provoke the degradation of stratospheric ozone and there is thus a need to provide aerosol formulations for medicaments which employ so called “ozone-friendly” propellants.

A class of propellants which are believed to have minimal ozone-depleting effects in comparison to conventional chlorofluorocarbons comprise fluorocarbons and hydrogen-containing chlorofluorocarbons, and a number of medicinal aerosol formulations using such propellant systems are disclosed in, for example, EP 0372777, WO91/04011, WO91/11173, WO91/11495 and WO91/14422. These applications are all concerned with the preparation of pressurised aerosols for the administration of medicaments and seek to overcome the problems associated with the use of the new class of propellants, in particular the problems of stability associated with the pharmaceutical formulations prepared. The applications all propose the addition of one or more of adjuvants such as alcohols, alkanes, dimethyl ether, surfactants (including fluorinated and non-fluorinated surfactants, carboxylic acids, polyethoxylates etc) and even conventional chlorofluorocarbon propellants in small amounts intended to minimise potential ozone damage.

A group of fluorinated derivatives of lecithin are described in EP478686 (Alliance) and their use as surfactants in aerosol formulations is described in WO96/09816 (Glaxo). Fluorinated surfactants for use with hydrofluoroalkanes are also described in U.S. Pat. No. 5,126,123, WO91/11173, WO91/14422, WO92/00062 and WO96/09816.

Unfortunately, fluorinated surfactants, especially perfluorinated surfactants which have desirable properties with respect to formulation stability often suffer from the problem of bioaccumulation because the body has difficulty in metabolising highly fluorinated molecules. Therefore, it has been difficult to find a suitable fluorinated surfactant with all the desirable characteristics and minimal undesirable characteristics.

Surprisingly, the inventors have now found a particular group of fluorinated surfactants suitable for use in the preparation of aerosol suspensions formulations which have useful suspension stablising properties, and can be advantageous in terms of providing reproducible dosing, reducing drug deposition, increasing shelf life and like, and which are expected to be less susceptible to bioaccumulation.

Thus, in one aspect the invention provides a compound of the general formula (I)

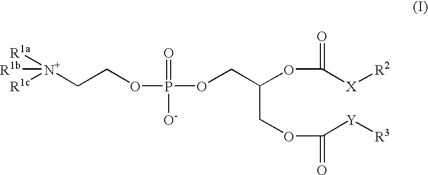

or a salt thereof, wherein:

R^{1a} represents C₁₋₃ alkyl;

R^{1b} represents C₁₋₃ alkyl

R^{1c} represents C₁₋₃ alkyl;

R² represents C₁₋₅ fluoroalkyl;

R³ represents C₁₋₅ fluoroalkyl;

X represents —C₁₋₆ alkylene-;

Y represents —C₁₋₆ alkylene-;

with the proviso that each C₁₋₆ fluoroalkyl group contains 3 or fewer consecutive perfluorocarbon atoms.

Preferably R^{1a} represents —CH₃.

Preferably R^{1b} represents —CH₃.

Preferably R^{1c} represents —CH₃.

Preferably R² represents C₁₋₃ fluoroalkyl, more preferably C₁₋₂ fluoroalkyl, especially —CF₂CF₃.

Preferably R³ represents C₁₋₃ fluoroalkyl, more preferably C₁₋₂ fluoroalkyl, especially —CF₂CF₃.

Preferably R² represents the same as R³.

Preferably X represents —C₁₋₃ alkylene-, especially —C₂₋₃ alkylenl-, particularly —CH₂CH₂—.

Preferably Y represents —C₁₋₃ alkylene-, especially —C₂₋₃ alkylene-, particularly —CH₂CH₂—.

Preferably X represents the same as Y.

In another aspect the invention provides a pharmaceutical aerosol formulation which comprises particulate medicament, a fluorocarbon or hydrogen-containing chlorofluorocarbon propellant and a compound of general formula (I).

Suitable salts of the compounds of formula (I) include physiologically acceptable salts and salts which may not be physiologically acceptable but may be useful in the preparation of compounds of formula (I) and physiologically acceptable salts thereof. If appropriate, acid addition salts may be derived from inorganic or organic acids, for example hydrochlorides, hydrobromides, sulphates, phosphates, acetates, benzoates, citrates, succinates, lactates, tartrates, fumarates, maleates, 1-hydroxy-2-naphthoates, palmoates, methanesulphonates, formates or trifluoroacetates.

Certain compounds of formula (I) may contain one or more chiral centres. It will be understood that compounds of formula (I) include all optical isomers of the compounds of formula (I) and mixtures thereof, including racemic mixtures thereof.

The surfactant compounds employed for the preparation of formulations according to the present invention are effective stabilisers at low concentrations relative to the amount of medicament and are adequately soluble in HFA propellants. Thus, the amount of surfactant employed is desirably in the range of 0.005 to 20% w/w, particularly 0.05 to 20% w/w, more particularly 0.05 to 15% w/w, even more particularly about 0.1 to about 10% w/w, and preferably 0.5 to about 10% w/w, relative to the medicament.

The particle size of the particulate (e.g. micronised) medicament should be such as to permit inhalation of substantially all of the medicament into the lungs upon administration of the aerosol formulation and will thus be less than 100 microns, desirably less than 20 microns, and preferably in the range 1-10 microns, e.g. 1-5 microns.

The final aerosol formulation desirably contains 0.00510% w/w, preferably 0.005-5% w/w, especially 0.01-1.0% w/w, of medicament relative to the total weight of the formulation.

Medicaments which may be administered in aerosol formulations according to the invention include any drug useful in inhalation therapy and which may be presented in a form which is substantially completely insoluble in the selected propellant. Appropriate medicaments may thus be selected from, for example, analgesics, e.g. codeine, dihydromorphine, ergotamine, fentanyl or morphine; anginal preparations, e.g. diltiazem; antiallergics, e.g. cromoglycate (e.g. as sodium salt), ketotifen or nedocromil (e.g. as sodium salt); anti-infectives e.g. cephalosporins, penicillins, streptomycin, sulphonamides, tetracyclines and pentamidine; anti-histamines, e.g. methapyrilene; anti-inflammatories, e.g. bedomethasone (e.g. as dipropionate), fluticasone (e.g. as propionate), flunisolide, budesonide, rofleponide, mometasone furoate, ciclesonide, triamcinolone acetonide or 6α, 9α-difluoro-11β-hydroxy-16α-methyl-3-oxo-17α-propionyloxy-androsta-1,4-diene-17β-carbothioic acid S-(2-oxo-tetrahydro-furan-3-yl) ester; anti-tussives, e.g. noscapine; bronchodilators, e.g. albuterol (e.g. as free base or sulphate), salmeterol (e.g. as xinafoate), ephedrine, adrenaline, fenoterol (e.g. as hydrobromide), formoterol (e.g. as fumarate), isoprenaline, metaproterenol, phenylephrine, phenylpropanolamine, pirbuterol (e.g. as acetate), reproterol (e.g. as hydrochloride), rimiterol, terbutaline (e.g. as sulphate), isoetharine, tulobuterol, 4-hydroxy-7-[2-[[2-[[3-(2-phenylethoxy)pro-pyl]sulfonyl]ethyl]amino]ethyl-2(3H)-benzothiazolone; diuretics, e.g., amiloride; anticholinergics, e.g. ipratropium (e.g. as bromide), tiotropium, atropine or oxitropium; hormones, e.g. cortisone, hydrocortisone or prednisolone; xanthines, e.g. aminophylline, choline theophyllinate, lysine theophyllinate or theophylline; therapeutic proteins and peptides, e.g. insulin or glucagon. It will be clear to a person skilled in the art that, where appropriate, the medicaments may be used in the form of salts, (e.g. as alkali metal or amine salts or as acid addition salts) or as esters (e.g. lower alkyl esters) or as solvates (e.g. hydrates) to optimise the activity and/or stability of the medicament and/or to minimise the solubility of the medicament in the propellant. It will be further clear to a person skilled in the art that where appropriate the medicaments may be used in the form of a pure isomer, for example, R-albuterol or RR-formoterol.

Particularly preferred medicaments for administration using aerosol formulations in accordance with the invention include anti-allergics, bronchodilators and anti-inflammatory steroids for use in the treatment of respiratory disorders such as asthma, COPD or rhinitis by inhalation therapy, for example cromoglycate (e.g. as sodium salt), albuterol (e.g. as free base or the sulphate), salmeterol (e.g. as xinafoate), formoterol (e.g. as fumarate), terbutaline (e.g. as sulphate), reproterol (e.g. as hydrochloride), a beclomethasone ester (e.g. as diproprionate), a fluticasone ester (e.g. as propionate). Salmeterol, especially salmeterol xinafoate, albuterol sulphate, fluticasone propionate, beclomethasone dipropionate and physiologically acceptable salts and solvates thereof are especially preferred.

It will be appreciated by those skilled in the art that the aerosol formulations according to the invention may, if desired, contain a combination of two or more active ingredients. Thus suitable combinations include bronchodilators (e.g. albuterol or isoprenaline) in combination with an anti-inflammatory steroid (e.g. beclomethasone ester); a bronchodilator in combination with an anti-allergic (e.g. cromoglycate). Exemplary combinations also include: ephedrine and theophylline; fenoterol and ipratropium (e.g. as bromide); isoetharine and phenylephrine; albuterol (e.g. as free base or as sulphate) and beclomethasone ester (e.g. as dipropionate); budesonide and formoterol (e.g. as fumarate) which is of particular interest; and salmeterol (particularly as salmeterol xinafoate) and fluticasone ester (e.g. as propionate) also of particular interest.

The propellants for use in the invention may be any fluorocarbon or hydrogen-containing chlorofluorocarbon or mixtures thereof having a sufficient vapour pressure to render them effective as propellants. Preferably the propellant will be a non-solvent for the medicament. Suitable propellants include, for example, C₁₋₄hydrogen-containing chlorofluorocarbons such as CH₂ClF, CClF₂CHClF, CF₃CHClF, CHF₂CClF₂, CHClFCHF₂, CF₃CH₂Cl and CClF₂CH₃; C₁₋₄hydrogen-containing fluorocarbons such as CHF₂CHF₂, CF₃CH₂F, CHF₂CH₃ and CF₃CHFCF₃; and perfluorocarbons such as CF₃CF₃ and CF₃CF₂CF₃.

Where mixtures of the fluorocarbons or hydrogen-containing chlorofluorocarbons are employed they may be mixtures of the above identified compounds, preferably binary mixtures, with other fluorocarbons or hydrogen-containing chlorofluorocarbons for example CHClF₂, CH₂F₂ and CF₃CH₃. Particularly preferred as propellants are C₁₋₄hydrogen-containing fluorocarbons such as 1,1,1,2-tetrafluoroethane (CF₃CH₂F) and 1,1,1,2,3,3,3-heptafluoro-n-propane (CF₃CHFCF₃) or mixtures thereof. Preferably a single fluorocarbon or hydrogen-containing chlorofluorocarbon is employed as the propellant e.g. 1,1,1,2-tetrafluoroethane (HFA 134a) or 1,1,1,2,3,3,3heptafluoro-n-propane (HFA 227), especially 1,1,1,2-tetrafluoroethane.

It is desirable that the formulations of the invention contain no components, which may provoke the degradation of stratospheric ozone. In particular it is desirable that the formulations are substantially free of chlorofluorocarbons such as CCl₃F, CCl₂F₂ and CF₃CCl₃.

The propellant may additionally contain a volatile adjuvant such as a saturated hydrocarbon, for example, propane, n-butane, isobutane, pentane and isopentane or a dialkyl ether, for example, dimethyl ether. In general, up to 50% w/w of the propellant may comprise a volatile hydrocarbon, for example, 1 to 30% w/w. However, formulations which are substantially free of volatile adjuvants are preferred. In certain cases, it may be desirable to include appropriate amounts of water, which can be advantageous in modifying the dielectric properties of the propellant.

Polar adjuvants which may if desired, be incorporated into the formulations according to the present invention include e.g. C₂₋₆ aliphatic alcohols and polyols such as ethanol, isopropanol and propylene glycol and mixtures thereof. Preferably ethanol will be employed. In general only small quantities (e.g. 0.05 to 3.0% w/w) of polar adjuvants are required and the use of quantities in excess of 5% whv may disadvantageously tend to dissolve the medicament. Formulations preferably contain less than 1% w/w, e.g. about 0.1% w/w of polar adjuvant. Polarity may be determined, for example, by the method described in European Patent Applicaffon Publication No. 0327777.

However, as the compounds of formula (I) are adequately soluble in the fluorocarbon or hydrogen-containing chlorofluorocarbon propellant the need to use a polar adjuvant is obviated. This is advantageous as polar adjuvants especially ethanol are not suitable for use with all patient groups. Formulations containing a compound of formula (I) which avoid use of a polar adjuvant are preferred.

In addition to the surfactant compounds of general formula (I), the formulations according to the present invention may optionally contain one or more further ingredients conventionally used in the art of pharmaceutical aerosol formulation. Such optional ingredients include, but are not limited to taste masking agents, one or more sugars, buffers, antioxidants, water and chemical stabilisers.

Aptly, the aerosol formulations according to the present invention may contain 0.0001 to 50% w/w, preferably 0.001 to 20, for example 0.001 to 1% of sugar relative to the total weight of the formulation. Generally the ratio of medicament:sugar falls within the range of 1:0.01 to 1:100 preferably 1:0.1 to 1:10. Typical sugars which may be used in the formulations, for example, include sucrose, lactose and dextrose, preferably lactose, and reducing sugars such as mannitol and sorbitol, and sugars may be in micronised or milled form. Most preferably the aerosol formulations according to the present invention will be substantially free of sugar.

Surfactant compounds according to the present invention can be prepared by techniques well known in the art as can be seen, for example, by reference to EP478686. A suitable process for preparing compounds of formula (I) comprises:

(a) reacting compound of formula (II)

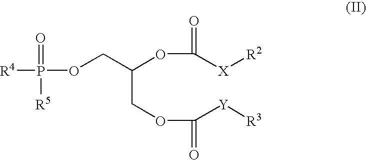

wherein R², R³ X and Y are as defined above and R⁴ and R⁵ represent a halogen atom (e.g. bromo or chloro, preferably chloro) with a compound of formula (III)

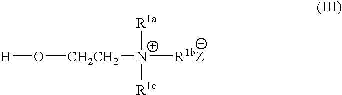

where Z is a negatively charged counter ion, such as a halide or arylsulphonyloxy group, such as mesylate or tosylate, followed by work-up with a hydrolysing agent, such as water; or

(b) reacting a compound of formula (IV)

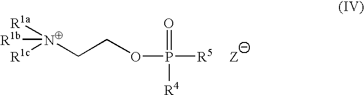

wherein R^{1a}, R^{1b}, R^{1c}, R⁴, R⁵ and Z are as defined above, with a compound of formula (V)

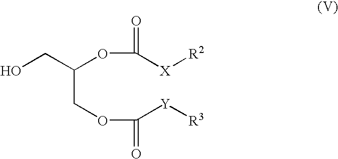

or a salt thereof wherein R², R³ X and Y are as defined above followed by work-up with a hydrolysing agent such as water; or

(c) reacting a compound of formula (VI)

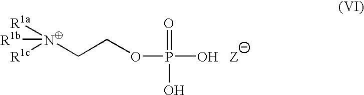

wherein R^{1a}, R^{1b}, R^{1c} and Z are as defined above, with a compound of formula (VII)

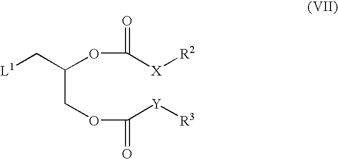

wherein R², R³ X and Y are as defined above and L¹ represents a leaving group such as halogen (e.g. chlorine).

Suitably process (a) may be carried out in the presence of a chlorinated organic solvent, such as chloroform or the like, and a basic medium, e.g. pyridine or the like. R⁴ and R⁵ will typically represent chlorine.

Conditons described above for process (a) are also suitable for process (b) and process (c).

Compounds of formula (II) may be prepared by treating an alcohol of formula (V) as defined above with phosphorylating agent, for example, POCl₃. The reaction may be performed in the presence of a sterically hindered base such as triethylamine in a solvent such as ether or chloroform at a non-extreme temperature e.g. −30 to 30° C. such as −20° C. or 0-5° C.

Compounds of formula (III) may be prepared from an amine of formula (VIII)

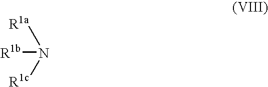

wherein R^{1a}, R^{1b} and R^{1c} are as defined above with ethylene oxide or chlorohydrin. When the reaction is performed using ethylene oxide an appropriate counter ion will be introduced during the work up.

Compounds of formula (IV) may be prepared by reacting a compound of formula (III) with a phosphorylating agent, such as POCl₃ under conditions described above for the preparation of compounds of formula (II).

Compounds of formula (V) may be prepared from glycerol or a protected derivative thereof or from epibromohydrin by known methods.

Compounds of formula (VI) may be prepared by reacting a compound of formula (III) with POCl₃ followed by hydrolysis or by heating said compounds with pyrophosphoric acid or polyphosphoric acid. Preferably in these reactions Z will represent chloro.

Compounds of formula (VII) can be prepared by reacting the alcohol of formula (V) with a halogenating agent or agent capable of converting the hydroxyl into O-tosyl, O-triflyl or O-mesyl.

Compounds of formula (II), (V) and (VII) are new and form an aspect of the invention.

The formulations of the invention may be prepared by dispersal of the medicament and surfactant in the selected propellant in an appropriate container, e.g. with the aid of sonication or a high-shear mixture. The process is desirably carried out under controlled humidity conditions to obviate any adverse effects of moisture on the suspension stability.

The chemical and physical stability and the pharmaceutical acceptability of the aerosol formulations according to the invention may be determined by techniques well known to those skilled in the art. Thus, for example, the chemical stability of the components may be determined by HPLC assay, for example, after prolonged storage of the product. Physical stability data may be gained from other conventional analytical techniques such as, for example, by leak testing, by valve delivery assay (average shot weights per actuation), by dose reproducibility assay (active ingredient per actuation) and spray distribution analysis.

The suspension stability of aerosol formulations may be measured by conventional techniques, for example, by measuring flocculation size distribution using a back light scattering instrument or by measuring particle size distribution by cascade impaction or by the “twin impinger” analytical process. As used herein reference to the “twin impinger” assay means “Determination of the deposition of the emitted dose in pressurised inhalations using apparatus A” as defined in British Pharmacopaeia 1988, pages A204-207, Appendix XVII C. Such techniques enable the “respirable fraction” of the aerosol formulations to be calculated. One method used to calculate the “respirable fraction” is by reference to the “fine particle fraction” which is the amount of active ingredient collected in the lower impingement chamber per actuation expressed as a percentage of the total amount of active ingredient delivered per actuation using the twin impinger method described above.

The formulations according to the invention may be filled into canisters suitable for delivering pharmaceutical aerosol formulations. Canisters generally comprise a container capable of withstanding the vapour pressure of the propellant used such as a plastic or plastic-coated glass bottle or preferably a metal can, for example, an aluminium can which may optionally be anodised, lacqueroated and/or plastic-coated (e.g. incorporated herein by reference WO96/32099), which container is closed with a metering valve. The metering valves are designed to deliver a metered amount of the formulation per actuation and incorporate a gasket to prevent leakage of propellant through the valve. The gasket may comprise any suitable elastomeric material such as, for example, low density polyethylene, chlorobutyl, black and white butadiene-acrylonitrile rubbers, butyl rubber and neoprene. EPDM rubber may also be advantageous. Suitable valves are commercially available from manufacturers well known in the aerosol industry, for example, from Valois, France (e.g. DF10, DF30, DF60), Bespak pic, UK (e.g. BK300, BK357) and 3M-Neotechnic Ltd, UK (e.g. Spraymiser™).

Conventional bulk manufacturing methods and machinery well known to those skilled in the art of pharmaceutical aerosol manufacture may be employed for the preparation of large scale batches for the commercial production of filled canisters. Thus, for example, in one bulk manufacturing method a metering valve is crimped onto an aluminium can to form an empty canister. The particulate medicament is added to a charge vessel and liquefied propellant is pressure filled through the charge vessel into a manufacturing vessel, together with liquefied propellant containing the surfactant. The drug suspension is mixed before recirculation to a filling machine and an aliquot of the drug suspension is then filled through the metering valve into the canister. In an altemative process, an aliquot of the liquefied formulation is added to an open canister under conditions which are sufficiently cold to ensure the formulation does not vaporise, and then the metering valve is crimped into the canister.

Typically, in batches prepared for pharmaceutical use, each filled canister is check-weighed, coded with a batch number and packed into a tray for storage before release testing.

Each filled canister is conveniently fitted into a suitable channelling device prior to use to form a metered dose inhaler for administration of the medicament into the lungs or nasal cavity of a patient. Suitable channelling devices comprise, for example, a valve actuator and a cylindrical or cone-like passage through which medicament may be delivered from the filled canister via the metering valve to the nose or mouth of a patient e.g. a mouthpiece actuator. Metered dose inhalers are designed to deliver a fixed unit dosage of medicament per actuation or “puff”, for example, in the range of 10 to 5000 microgram medicament per puff.

Administration of medicament may be indicated for the treatment of mild, moderate or severe acute or chronic symptoms or for prophylactic treatment. It will be appreciated that the precise dose administered will depend on the age and condition of the patient, the particular particulate medicament used and the frequency of administration and will ultimately be at the discretion of the attendant physician. When combinations of medicaments are employed the dose of each component of the combination will in general be that employed for each component when used alone. Typically, administration may be one or more times, for example, from 1 to 8 times per day giving, for example, 1,2,3 or 4 puffs each time.

Suitable daily doses, may be, for example in the range 50 to 200 microgram of salmeterol (e.g. as xinfoate), 100 to 1000 microgram of salbutamol (e.g. as sulphate), 50 to 2000 microgram of fluticasone propionate or 100 to 2000 microgram of beclomethasone dipropionate, depending on the severity of the disease.

Thus, for example, each valve actuation may deliver 25 microgram salmeterol, 100 microgram salbutamol, 25, 50, 125 or 250 microgram fluticasone propionate or 50, 100, 200 or 250 microgram beclomethasone dipropionate. Typically each filled canister for use in a metered dose inhaler contains 100, 120, 160 or 240 metered doses or puffs of medicament.

A suitable method for accessing the bioaccumulation of compounds according to the invention includes intravenously dosing a Wistar Han rat (B30603) with 10 mg per Kg of the desired compound in a 25% DMSO 75% saline carrier. Plasma samples may then be taken at desired intervals, for example 10, 20, 40, 90 150 minutes, and 6, 24 and 32 hours. The samples may be prepared by extraction using protein precipitation and then analysed using high-performance liquid chromatography using a suitable detector such as a liquid chromatography tandem mass spectrometer. The half-life of the compound can then be calculated by known methods.

The filled canisters and metered dose inhalers described herein comprise further aspects of the present invention.

The invention also extends to use of a compound of formula (I) as a surfactant, especially in a pharmaceutical aerosol formulation comprising a fluorocarbon or hydrogen-containing chlorofluorocarbon propellant, such as 1,1,1,2-tetrafluoroethane and 1,1,1,2,3,3,3-heptafluoro-n-propane or mixtures thereof, and a particulate medicament.

A still further aspect of the present invention comprises a method of treating respiratory disorders such as, for example, asthma, which comprises administration by inhalation of an effective amount of a formulation as herein described. The words treating and treatment as used herein includes prophylactic treatment.

The following non-limiting Examples serve to illustrate the invention.

### EXAMPLES

### General Analytical Conditions for Retention Times (RT) Quoted

Retention times were determined using an lnertpak ODS 2 column (15 cm×4.6 mm ID, 5 micron) eluting with 0.05% trifluoracetic acid (TFA) in water (solvent A), and 0.05% TFA in acetonitrile (solvent B), using the following elution gradient 0-20 min 0-95% B, hold 5 min 95% B, at a flow rate of 1 ml/min. The detection method was Atmospheric Pressure Electrospray positive ion.

### Example 1

### Bis(4,4,5,5,5-pentafluopentanoyl)phosphatidylcholine

a) 4,4,5,5,5-Pentafluoropentanoic acid

4,4,5,5,5-Pentafluoropentanoic acid was synthesised by the method described in Organic Process Research and Development 1999, 3, 363-364.

b) 2-[(4,4,5,5,5-Pentafluoropentanoxyl)oxy]-1-{[(phenylmethyl)oxy]methyl}ethyl 4,4,5,5,5-pentafluoropentanoate

The product of step (a) (30 g) and carbonyl diimidazole (25.3 g) were dissolved in tetrahydrofuran (THF)(80 ml) and stirred at 48° C. for 1 hour. A solution of benzyl glycerol (12.8 g) and 1,8-diazabicyclo[5.4.0]undec-7-ene (23.8 g) in THF (10 ml) was added and the reaction was stirred at 50° C. for 2 hours, then at 20° C. for a further 12 hours. The reaction mixture was partitioned between methyl tert-butyl ether (600 ml) and 1M hydrochloric acid (600 ml). The organic layer was washed with water (600 ml), saturated sodium bicarbonate solution (600 ml), water (300 ml), brine (300 ml), dried and the solvent removed in vacuo to give the title compound (38 g) as an orange oil. Mass spectrum m/z 548 [MNH₄⁺]

c) 2-Hydroxy-1-{[(4,4,5,5,5-pentafluoropentanoyl)oxy]methyl}ethyl 4,4,5,5,5-pentafluoro-pentanoate

The product of step (b) (20 g) was dissolved in THF(200 ml) and 10% Pd/C (2 g) was added. The reaction was placed under an atmosphere of hydrogen and stirred at 20° C. for 15 hours. The reaction mixture was filtered through a bed of celite and the solvent was removed in vacuo. Purification by column chromatography on silica gel (Biotage) eluting with 3:1 cyclohexane:ethyl acetate gave the title compound (15 g) as a clear oil.

Mass spectrum m/z 458 [MNH₄⁺]

d) Bis(4,4,5,5,5-pentafluopentanoyl)phosphatidylcholine

A solution of phosphorus oxychloride (1.8 ml) and triethylamine (2.9 ml) in isopropyl ether (10 ml) was stirred at −20° C. To this mixture a solution of the product of step (c) (8 g) in isopropyl ether (50 ml) was added, keeping the temperature below −15° C. Once the addition was complete the reaction was allowed to warm to 20° C., filtered and the solvent removed in vacuo. The residue was dissolved in chloroform (100 ml) and cooled to 0° C. Choline tosylate (5 g) was added, followed by a solution of pyridine (10 ml) in chloroform (20 ml). The resulting suspension was stirred at 20° C. for 15 hours, then water (5 ml) was added and the reaction stirred at 20° C. for a further 5 hours. The reaction mixture was added to a suspension of TMD-8 ion exchange resin (100 g) in ethanol (100 ml) and stirred at 20° C. for 2 hours. The mixture was filtered and the solvent removed in vacuo to give the title compound.

Mass spectrum m/z 606 [MH⁺]RT 13.4 minutes.

Experimental Data

Sample Preparation

Salmeterol xinafoate formulations in HFA 134a (1,1,1,2-tetrafluoroethane), of strength 25 μg per actuation, and 10% w/w (relative to drug) of the surfactant compound of Example 1 (Bis(4,4,5,5,5-pentafluopentanoyl)phosphatidylcholine) were prepared using salmeterol xinafoate (5.8 mg), HFA 134a (12 g) and the relevant compound (0.58 mg). The control was prepared without the addition of a surfactant.

Particle Size Data

Table 1 shows mean particle size data determined by image analysis using a Galai CIS-100 particle size analyser for sample formulations prepared as described above. In this measurement, particle size is represented as the equivalent diameter of a circle of equal area to the object. The mean is the average of 4 determinations. The particle size measurement was obtained by transferring the suspensions to a presurised cell, and video-imaging the sample under shear via a microscope objective.

The equivalent diameter is defined as the diameter of a circle of equal area to the object.

$$Equivalent Diameter = \sqrt{\frac{Area}{π}}$$

The mean equivalent diameter can be weighted by number, length or volume. e.g. For three particles with equivalent diameters of x, y and z:

$$Mean Number weighted diameter = ( \frac{1}{3} ) x + ( \frac{1}{3} ) y + ( \frac{1}{3} ) z$$

$$Mean Length weighted diameter = ( \frac{x}{x + y + z} ) x + ( \frac{y}{x + y + z} ) y + ( \frac{z}{x + y + z} ) z$$

The data shows that the surfactant compound of Example 1 has suspension stabilising properties, thereby discouraging flocculation of drug particles. This is seen by the marked reduction in average particle size (“mean length weighted diameter”) when the said compound is incorporated into the formulation.

TABLE 1
Particle Size Data

| | Mean Length | | Relative |
|---|---|---|---|
| | weighted | Standard Deviation | Standard |
| | diameter μm | μm | Deviation |
| Control | 26.6 | 0.8 | 3.1 |
| Example 1 | 13.3 | 1.3 | 9.7 |

Andersen Cascade Impaction Data

The formulation, the preparation of which is described above, was profiled using an Andersen Cascade Impactor. Ten actuations at “beginning of use” (BoU) were collected in the impactor from an inhaler after 4 priming actuations were fired to waste. The drug delivered was then quantified by HPLC analysis. Testing was performed intially (following sample preparation) and then after inhalers had been stored at 40° C./75% RH for 8 weeks and for 3 months. The results, at each timepoint, in Table 2 are shown as the mean analysis of 3 cans/inhalers.

The profile obtained was used to determine total dose emitted dose (ex-valve and ex-actuator) and the fine particle mass (FPM, defined as the sum of stages 3-5). The percentage fine particle mass expresses the FPM as a percentage of the total dose emitted (ex-valve). The FPM is used as a measure of the proportion of the drug likely to reach the therapeutic target in the lungs.

The data shows, that in the presence of the surfactant compound of Example 1, there is an increase in both the absolute doses emitted and the absolute FPM. There is also a significant increase in the percentage FPM.

TABLE 2
Total Ex-Valve & Ex-actuator Emitted Dose and FPM Data Using
Cascade Impaction

| | | Total Dose | | | |
|---|---|---|---|---|---|
| | | Emitted | Total Dose | | |
| | | (Ex-Valve) | Emitted (Ex- | FPM | |
| | Timepoint | μg | Actuator) μg | μg | % FPM |
| Control | Initial | 21.0 | 17.9 | 8.9 | 42.3 |
| | 8 week | 22.1 | 18.9 | 9.1 | 41.1 |
| | 3 month | 21.9 | 17.5 | 7.7 | 35.2 |
| Example 1 | Initial | 23.3 | 20.0 | 11.1 | 47.7 |
| | 8 week | 23.5 | 19.5 | 10.4 | 44.2 |
| | 3 month | 24.4 | 20.1 | 10.4 | 42.5 |

Content Uniformity

The content uniformity of the formulation, the preparation of which is described above, was assessed by dose through use testing. Testing was performed on 10 cans/inhalers at “beginning of use” (BoU) and “end of use” (EoU). After each inhaler had been primed (4 shots fired to waste), actuations 1 and 2 (BoU) were collected. The next 116 actuations of each inhaler were then fired to waste using an automated method and actuations 119 and 120 (EoU) collected.

Assesment of content uniformity was performed intially (following sample preparation) and then on a further 10 inhalers for each timepoint, 8 weeks and 3 months, which were stored at 40° C./75% RH. Mean results from the two BoU actuations (1+2 for 10 inhalers) and the two EoU actuations (119+120 for same 10 inhalers) at each timepoint together with the percentage relative standard deviation (% RSD) for the 10 cans are shown in Table 3.

The data shows, that in the presence of the surfactant compound of Eample 1, there is a decrease in the difference between the dose collected at the beginning and end of use. In the control there is a rise from beginning to end of use of between 6.3 and 7.8 μg. However, in the presence of the said surfactant compound this rise is reduced to between 2.9 and 3.9 μg and also there is a reduction in the percentage RSD at EoU for the 10 inhalers tested showing improved can to can reproducibility. The presence of the surfactant therefore improves the content uniformity of the inhaler.

TABLE 3

| | Control | | Example 1 | |
|---|---|---|---|---|
| | BoU dose | EoU dose | BuO dose | EuO dose |
| Timepoint | μg | μg | μg | μg |
| Initial | 17.7 | 24.0 | 19.6 | 23.3 |
| | (2.4% RSD) | (7.6% RSD) | (4.1% RSD) | (4.6% RSD) |
| 8 Week | 17.3 | 23.9 | 20.3 | 24.2 |
| | (3.6% RSD) | (5.8% RSD) | (1.8% RSD) | (2.7% RSD) |
| 3 Month | 17.7 | 25.5 | 19.7 | 22.6 |
| | (4.9% RSD) | (7.8% RSD) | (2.3% RSD) | (3.2% RSD) |

It will be understood that the present disclosure is for the purpose of illustration only and the invention extends to modifications, variations and improvements thereto which will be within the ordinary skill of the person skilled in the art.

Throughout the specification and the claims which follow, unless the context requires otherwise, the word ‘comprise’, and variations such as ‘comprises’ and “comprising”, will be understood to imply the inclusion of a stated integer or step or group of integers but not to the exclusion of any other integer or step or group of integers or steps.

1. A compound of formula (I) 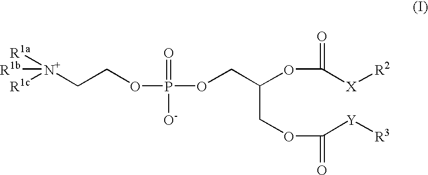
or a salt thereof, wherein:
R^{1a} represents C₁₋₃ alkyl;
R^{1c} represents C₁₋₃ alkyl;
R^{1c} represents C₁₋₃ alkyl;
R² represents C₁₋₅ fluoroalkyl;
R³ represents C₁₋₅ fluoroalkyl;
X represents —C₁₋₆ alkylene-;
Y represents —C₁₋₆ alkylene-;
with the proviso the each C₁₋₅ fluoroalkyl contains 3 or fewer consecutive perfluorocarbon atoms.

2. A compound according to claim 1 wherein R^{1a} represents methyl.

3. A compound according to claim 1 wherein R^{1b} represents methyl.

4. A compound according to claim 1 wherein R^{1c} represents methyl.

5. A compound according to claim 1 wherein R² represents C₁₋₃ fluoroalkyl.

6. A compound according to claim 5 wherein R² represents —CF₂CF₃.

7. A compound according to claim 1 wherein R³ represents C₁₋₃ fluoroalkyl.

8. A compound according to claim 7 wherein R³ represents —CF₂CF₃.

9. A compound according to claim 1 wherein X represents —CH₂CH₂—.

10. A compound according to claim 1 wherein Y represents —CH₂CH₂—.

11. Bis(4,4,5,5,5-pentafluopentanoyl)phosphatidylcholine.

12. A pharmaceutical aerosol formulation, which comprises particulate medicament, a fluorocarbon or hydrogen-containing chlorofluorocarbon propellant, or mixtures thereof and a compound according to claim 1.

13. A pharmaceutical aerosol formulation according to claim 12 wherein the particulate medicament is salmeterol or a physiologically acceptable salt thereof.

14. A pharmaceutical aerosol formulation according to claim 13 which further comprises fluticasone propionate.

15-16. (Canceled).

17. A method of treating respiratory disorders which comprises administering by inhalation an effective amount of a formulation according to claim 12.

18. A compound of formula (II) 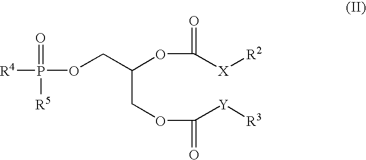
wherein
R² represents C₁₋₅ fluoroalkyl;
R³ represents C₁₋₅ fluoroalkyl;
R⁴ and R⁵ independently represent a halogen atom.
X represents —C₁₋₆ alkylene-;
Y represents —C₁₋₆ alkylene-;
with the proviso the each C₁₋₅ fluoroalkyl contains 3 or fewer consecutive perfluorocarbon atoms.

19. A compound of formula (V) 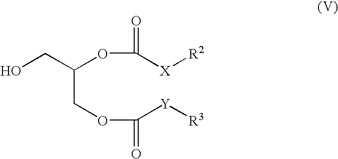
wherein
R² represents C₁₋₅ fluoroalkyl;
R³ represents C₁₋₅ fluoroalkyl;
X represents —C₁₋₆ alkylene-;
Y represents —C₁₋₆ alkylene-;
with the proviso the each C₁₋₅ fluoroalkyl contains 3 or fewer consecutive perfluorocarbon atoms.

20. A compound of formula (VII) 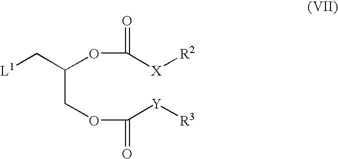
wherein
R² represents C₁₋₅ fluoroalkyl;
R³ represents C₁₋₅ fluoroalkyl;
X represents —C₁₋₆ alkylene-;
Y represents —C₁₋₆ alkylene-; and
L¹ represents a leaving group,
with the proviso the each C₁₋₅ fluoroalkyl contains 3 or fewer consecutive perfluorocarbon atoms.

21. A process for the preparation of a compound according to claim 1 which comprises:
(a) reacting compound of formula (II) 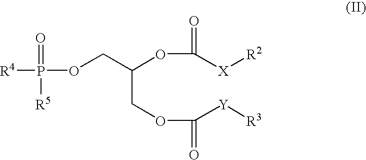
wherein R², R³ X and Y are as defined above for compounds of formula (I) and R⁴ and R⁵ represent a halogen atom with a compound of formula (III) 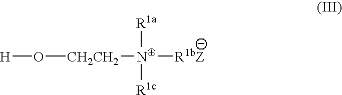
where Z is a negatively charged counter ion, followed by work-up with a hydrolysing agent; or
(b) reacting a compound of formula (IV) 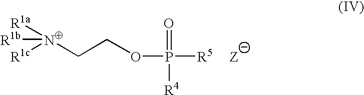
wherein R^{1a}, R^{1b}, R^{1c}, R⁴, R⁵ and Z are as defined above in process (a), with a compound of formula (V) 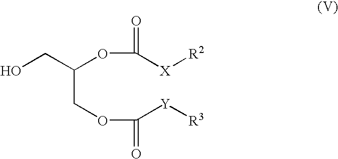
or a salt thereof wherein R², R³ X and Y are as defined above for compounds of formula (I) followed by work-up with hydrolysing agent; or
(c) reacting a compound of formula (VI) 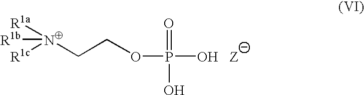
wherein R^{1a}, R^{1b}, R^{1c} and Z are as defined above for compounds of formula (I), with a compound of formula (VII) 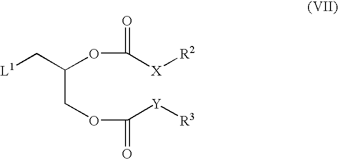
wherein R², R³ X and Y are as defined above for compounds and L¹ represents a leaving group.

22. (Canceled).

23. The method according to claim 17, wherein said respiratory disorder is asthma or COPD.
